(19) United States
(12) Patent Application Publication
(10) Pub. No.: US 2022/0359048 A1
(43) Pub. Date: Nov. 10, 2022

(54) AI AND ML ASSISTED SYSTEM FOR DETERMINING SITE COMPLIANCE USING SITE VISIT REPORT

(71) Applicant: IQVIA Inc., Danbury, CT (US)

(72) Inventors: Rajneesh Patil, Durham, NC (US); Virupaxkumar Bonageri, Bengaluru (IN); Gargi Shastri, Faridabad (IN)

(21) Appl. No.: 17/308,415

(22) Filed: May 5, 2021

(51) Int. Cl. G16H 10/20; G06F 40/30; G06N 20/00

(52) U.S. Cl. CPC G16H 10/20; G06F 40/30; G06N 20/00

(57) ABSTRACT

Methods and systems to automatically construct a clinical study site visit report (SVR), conduct the SVR, evaluate the SVR in real-time, and provide feedback while the SVR is being conducted. Responses to the SVR include user-selectable answers and natural language notes. Each response is evaluated as it is submitted based on a combination of pre-configured rules and a computer-trained model. If an anomaly is detected and is not already captured in the SVR, an alert is generated during performance of the SVR. The alert may include recommended remedial action.

### BACKGROUND

Clinical study sites are physical locations at which clinical studies (e.g., trials) are conducted.

Oversight visits to a study site may be required by contract (e.g., between a study sponsor and an investigator) and/or by government mandate. Oversight visits may include a pre-study visit, a site selection or site qualification visit, a site initiation visit, periodic monitoring visits, and a close-out visit. Periodic monitoring visits may be conducted to evaluate how a study is being conducted and to perform source document verification. Site visits help ensure that sites are compliant with regulations, that safety procedures are followed, that there are no significant protocol deviations, and that study documents are properly maintained.

Inquiries to be conducted during a site visit may be based on government requirements, sponsor requirements, study type, site features. Determination of the scope and detail of inquiries to be conducted during a site visit may involve considerable consultation amongst the sponsor, the investigator, and the site monitor.

Findings of a site visit are typically captured in a site visit report (SVR) by the site monitor. The site monitor may capture data in the form of short paragraphs or comments (i.e., free text/unstructured data). A SVR is manually reviewed by a specialist trained to identify issues from SVRs (e.g., protocol deviations or adverse events), and to provide remedial recommendations. A SVR may be part of a regulatory submission package for a clinical trial.

Under current practice, a site monitor and/or specialist may provide a SVR or SVR findings after a site visit has concluded (e.g., up to ten days later).

### SUMMARY

Disclosed herein are methods and systems to automate construction of a site visit report, automate evaluation of a site monitor's responses to questions of the SVR (i.e., user-selected answers and natural language notes) for compliance and/or adverse events, as the responses/notes are submitted by a site monitor, and to provide real-time feedback (e.g., while the site visit is being conducted and the site monitor is still on-site). Real-time feedback may include alerts when an adverse event is detected or when remedial action is recommended.

### BRIEF DESCRIPTION OF THE DRAWINGS

FIG. 1 is a diagram of a site visit report engine (SVR engine) configured to detect anomalies based on responses to questions of a SVR in real-time.

FIG. 2 is a diagram of another SVR engine configured to detect anomalies based on responses to questions of a site visit report in real-time.

FIG. 3 is an illustration of a SVR environment that includes an SVR engine accessible to multiple types of users.

FIG. 4 is a flowchart of a machine-implemented method of conducting and evaluating a site visit.

FIG. 5 is a flowchart of a method of configuring a SVR engine to evaluate responses to a SVR.

FIG. 6 is a flowchart of a method of configuring and using a SVR engine.

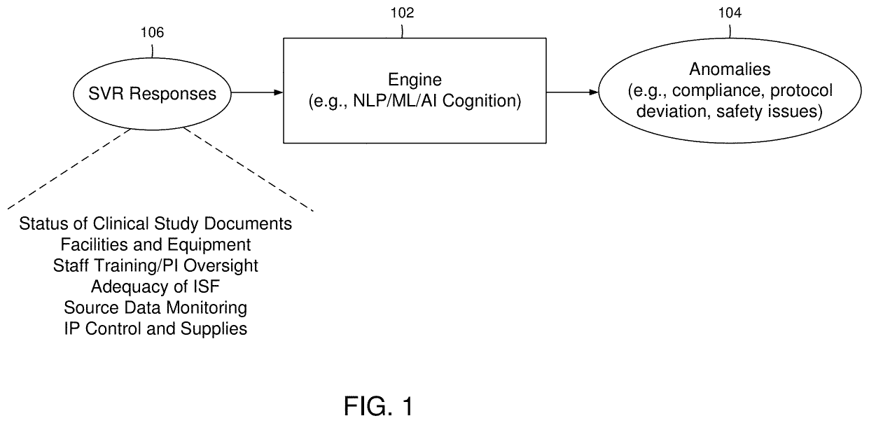

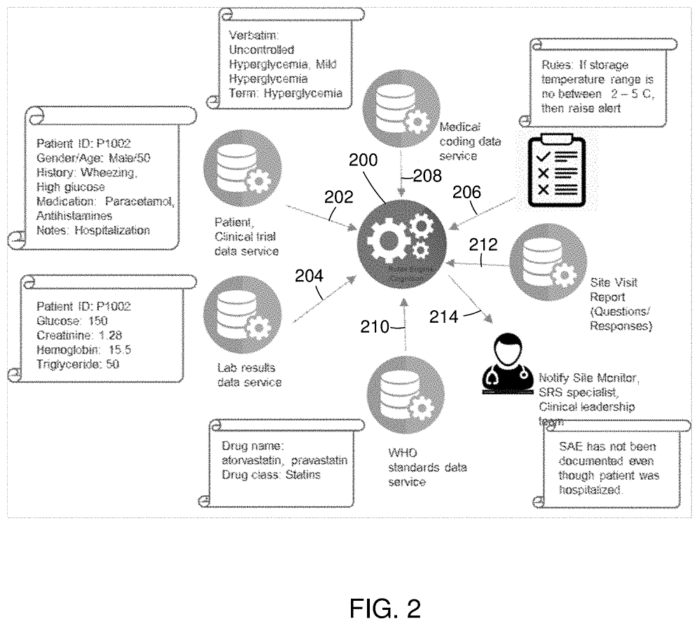

FIG. 2 illustrates a user interface window that includes a question presentation window, a pull-down list of user-selectable answers, and a comment window to receive natural language comments of a user.

FIG. 8 illustrates an example pop-up window.

FIG. 9 illustrates another example pop-up window.

FIG. 10 illustrates another user interface window that includes a question presentation window, user-selectable answers, and a comment window to receive natural language comments of a user.

FIG. 11 illustrates the user interface window of FIG. 10 and a corresponding feedback window to present ranking or scoring information based on user responses.

FIG. 12 illustrates a portion of an example SVR.

FIG. 13 is a block diagram of a computer-trainable model/function to correlate historical raw data (e.g., text analytics extracted from historical natural language notes associated with historical medical data, and answers of historical site visit reports), with corresponding supervisor-declared adverse events.

FIG. 14 illustrates a graph of adverse events and non-adverse events as determined by a supervisor and as determined by a model.

FIG. 15 is a block diagram of a computer system configured to evaluate responses to a site visit report (SVR) in real time.

In the drawings, the leftmost digit(s) of a reference number identifies the drawing in which the reference number first appears.

### DETAILED DESCRIPTION

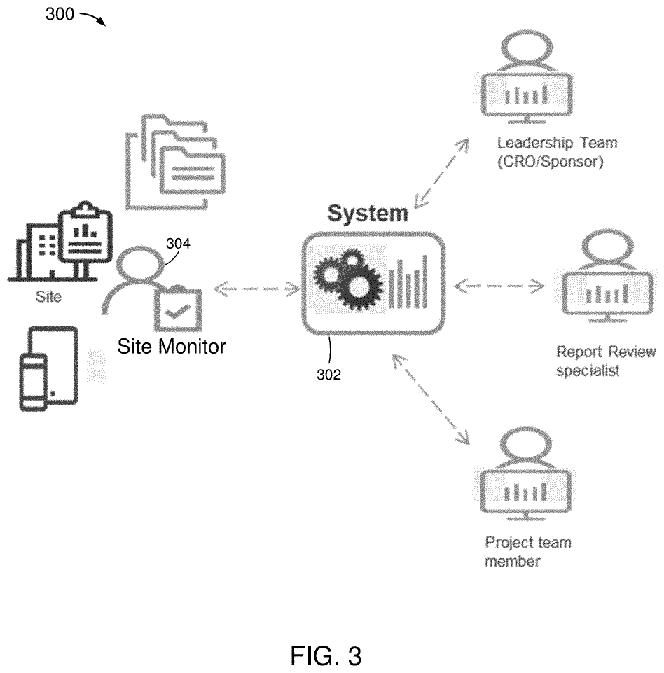

FIG. 3 is a diagram of a site visit report engine (SVR engine) 102 configured to detect anomalies 104 based on responses 106 to questions of a SVR, in real-time. SVR engine may be configured to detect anomalies 104 in real-time (i.e., as responses 106 are provided by a site monitor). In this way, remedial action may be recommended (e.g., by SVR engine 102) and initiated while the site monitor is on-site.

SVR engine 102 may include a rules-based engine and/or a cognition engine. SVR engine 102 may be configured to perform natural language processing (NLP), machine learning (ML), and/or artificial intelligence (AI).

NLP may include topic detection and/or sentiment analysis. NLP tasks may involve assigning annotation data such as grammatical information to words or phrases within a natural language expression. Different classes of machine-learning algorithms may be applied to NLP tasks. These algorithms may take a set of features generated from the natural language data as input. Some algorithms, such as decision trees, utilize hard if-then rules. Other systems use neural networks or statistical models which make soft, probabilistic decisions based on attaching real-valued weights to input features. These models can express the relative probability of multiple answers.

SVR engine 102 may be configured as described in one or more examples below. SVR engine 102 is not, however, limited to the following examples.

FIG. 2 is a diagram of a SVR engine 200 configured to detect anomalies based on responses to questions of a site visit report in real-time.

At 202, patient data is captured in electronic case forms by hospital staff, and trial data is captured in various systems such as a clinical trial management system and interactive web/voice response systems (e.g., to document notes, events like protocol deviations, and adverse events).

At 204, lab tests performed on patient is captured (e.g., in lab systems).

At 206, rules to be evaluated for every visit are configured and saved by medical experts.

At 208, a medical coding expert maps verbatim of adverse events, medical history, etc., into low level terminology (e.g., as specified in MedDRA hierarchy).

At 210, World Health Organization (WHO) dictionary standards are referred to, to identify drugs and their classification and to determine if they are permitted in trial. For example, statin usage is not permitted in certain trails.

At 212, a site monitor provides responses to questions and sub questions of a site visit report and adds notes when applicable. A note may explain, for example, that a subject was hospitalized for more than 24 hours, IP compliance for the subject is low, facilities at site are not adequate, site storage has temperature breach, site staff is not adequately trained, documents are missing in investigator site file (ISF), etcetera.

At 214, for each response captured in a SVR, SVR engine 200 performs an inspection to ensure that the response conforms to the data service of 202, 204, and/or 206.

Based on rules listed in service 206 and historical clinical data and documented events, SVR engine 200 performs subject data review activities using rule evaluation and machine learning algorithms encompassing supervised/unsupervised algorithms, natural language processing (NLP) for topic modelling. This may include, for example, subject eligibility to check if the subjects enrolled are of prescribed age group could be evaluated with simple rules.

Adverse prediction may be performed by looking at historical data and the actions taken by project team members in the past. If there are any recommended protocol deviations, adverse events being predicted by the system, SVR engine 200 may check if they are captured in the SVR. If there are any discrepancies, the site monitor, sponsor, and/or investigator may be notified based on the severity of the alert.

Related questions/responses in site visit report may be inspected collectively (holistic review) to ensure that there are no discrepancies. If a question related to IP compliance has notes captured by the Site Monitor that subject has not taken an investigational product (IP) as he was hospitalized for more than 24 hours, then there should be a severe adverse event recorded in the associated section. If there are discrepancies it will be flagged,

FIG. 3 is an illustration of a SVR environment 300 that includes an SVR engine 302 accessible to multiple types of users.

In the example of FIG. 3, a site monitor 304 is presented with list of questions in a mobile app that needs to be answered at a site visit. As site monitor 304 captures the information in the app, SVR engine 302 computes an alert if there are any discrepancies and compliance issues. Responses from site monitor 304 may be reviewed/evaluated in real time.

Alerts/recommendations may be reviewed/evaluated in real time by a report review specialist remotely, who may provide feedback in real-time so that site monitor can action on it while at site rather than waiting for next site visit. This improves cycle times and communication efficiency.

For example, when a response to a question related to informed consent is responded in the affirmative in the SVR, but the dates when the subjects signed the ICF's are not tracked, this may pose a risk around authenticity of the information or it could be typographical error. This may be flagged for further review.

A report review specialist may use real time analytics generated by SVR engine 302 to conduct holistic reviews. During the review, if it is observed that the site had too many protocol deviations, issues with the patient consenting, investigational product (IP) management, and compliance with investigator site file (ISF) is not satisfactory, then the report review specialist may immediately identify the inconsistency and escalate to a clinical operations team for appropriate management. This can help to avoid or prevent adverse regulatory and inspection findings.

SVR engine 302 may be able to highlight issues not just related to the site but also related to performance/management of a site monitor. For example, during prior monitoring visits, the site monitor may not have verified safety letters acknowledgement/signed by the principal investigator (PI) due to poor prioritization of the task during the monitoring visit. Another related example is due to process or system flaws, all safety letters may not be received by the site and those discrepancies can be identified by the site monitor during the monitoring visit. An operations oversight team may receive an alert so that they can provide appropriate training and mentoring to the site monitor. These tasks may be logged in SVR 302 for tracking and continuous process and delivery improvement.

A SVR engine may be configured to perform a method such as described in one or more examples below.

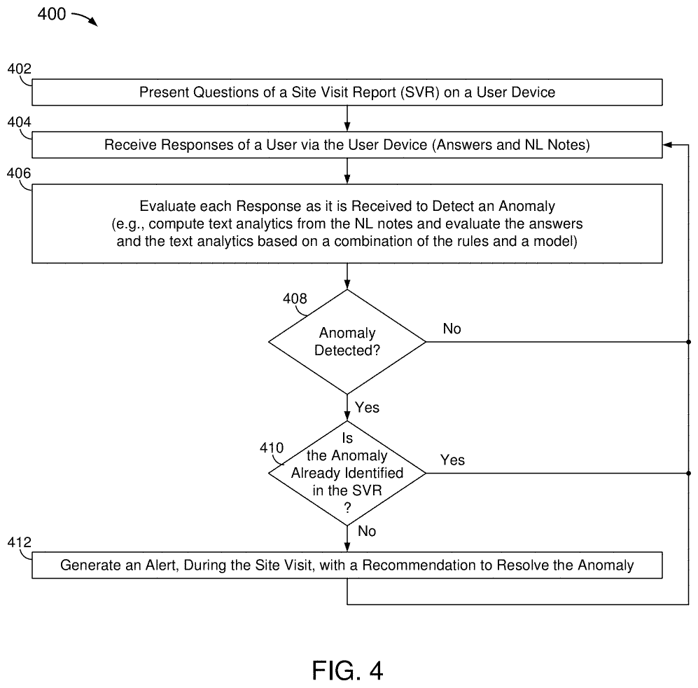

FIG. 4 is a flowchart of a machine-implemented method 400 of conducting and evaluating a site visit.

At 402, questions of a SVR are presented in a sequential fashion on a user device.

At 404, responses to the questions are received via the user device. The responses may include user-selectable answers and natural language notes of a user.

At 406, each response and/or a group of responses is evaluated as it is received from the user device to detect an anomaly in the clinical trial site visit. An anomaly may be defined as protocol deviation and/or an adverse event.

The evaluating at 406 may include computing text analytics from natural language notes of a user. Computing text analytics may include performing sentiment analytics and/or topical analytics.

The evaluating at 406 may further include evaluating user-selected answers and the text analytics based on a combination of pre-configured rules and a computer-trained model.

At 408, if no anomaly is detected, processing returns to 404.

At 410, if a detected anomaly has already been identified in the SVR (e.g., by a site monitor), processing returns to 404.

At 412, if a detected anomaly has not already been identified, an alert is generated. The alert may be generated while the site visit is ongoing. The alert may be directed to the site monitor and/or to a supervisor at a location that is remote from the site monitor. The alert may include a recommendation for remedial action, which may be initiated while the site monitor is on-site.

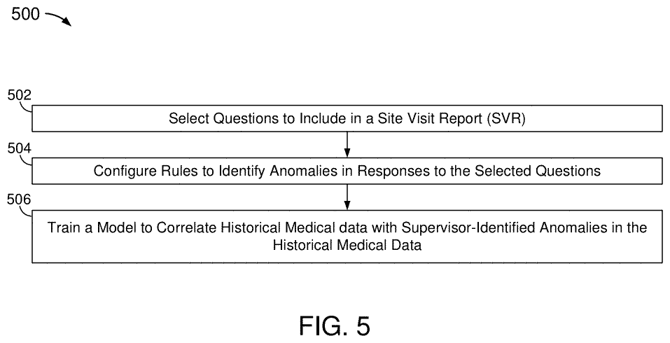

FIG. 5 is a flowchart of a method 500 of configuring a SVR engine to evaluate responses to a SVR. Method 500 may be used to configure a SVR engine to perform method 400. Method 500 is not, however, limited to the example of method 400. Nor is method 400 limited to the example of method 500.

At 502, questions to include in the SVR are selected. The questions may be selected from a configuration file (csv/excel/xml), based on features of a study, a site, and/or other factors0.

At 504, rules are configured to identify anomalies from responses to questions of a SVR (i.e., user-selected responses and/or natural language comments).

At 506, a model is trained to identify additional anomalies (e.g., adverse events) from site monitor responses. The model may be trained, for example, to correlate historical medical data to supervisor-identified anomalies in the historical medical data. Examples are provided further below with reference to FIGS. 13 and 14.

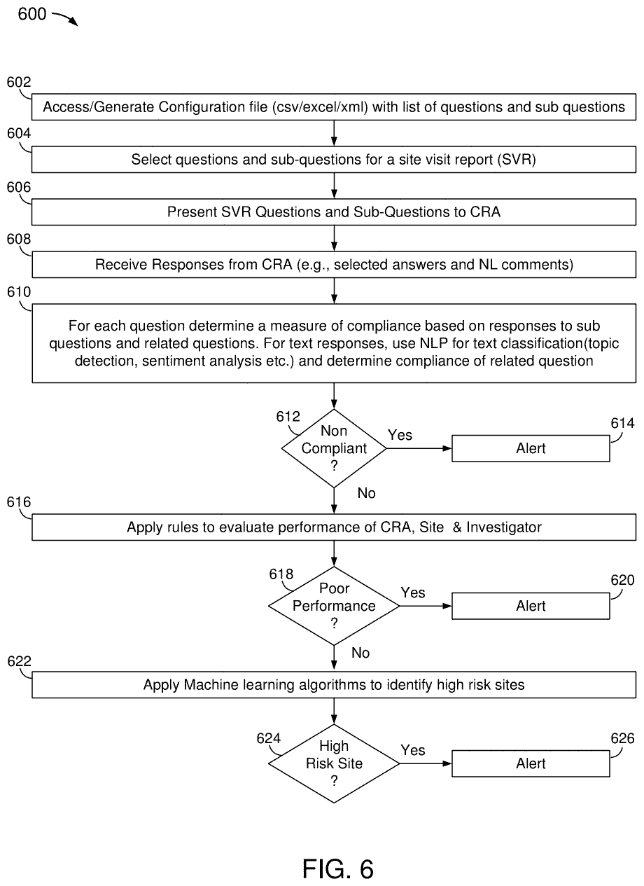

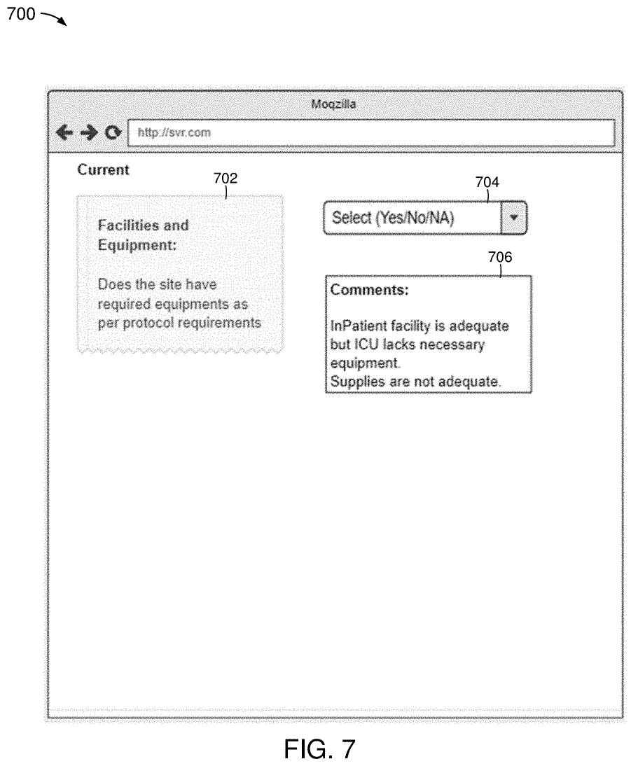

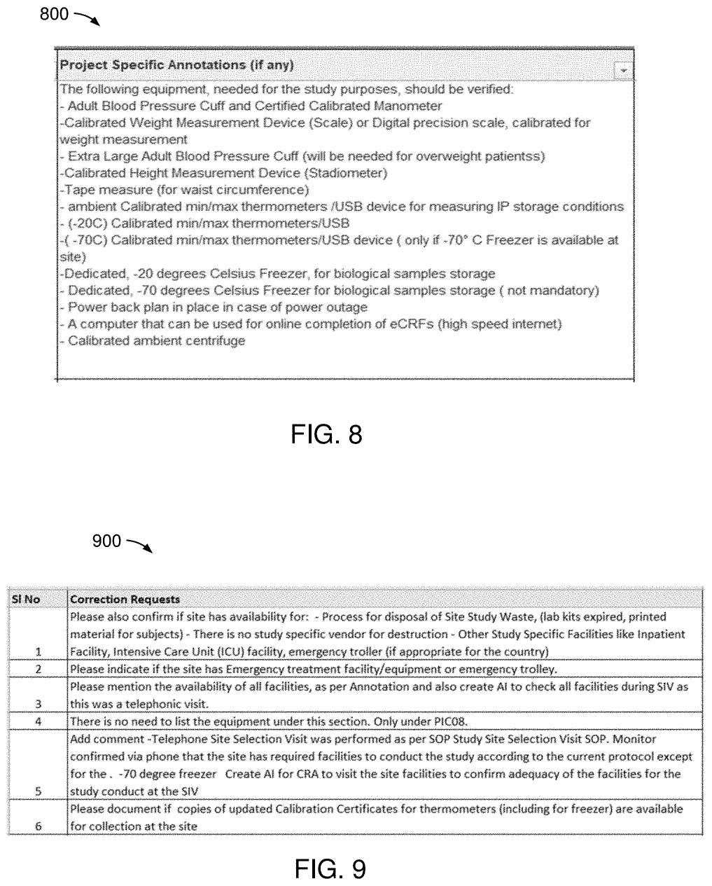

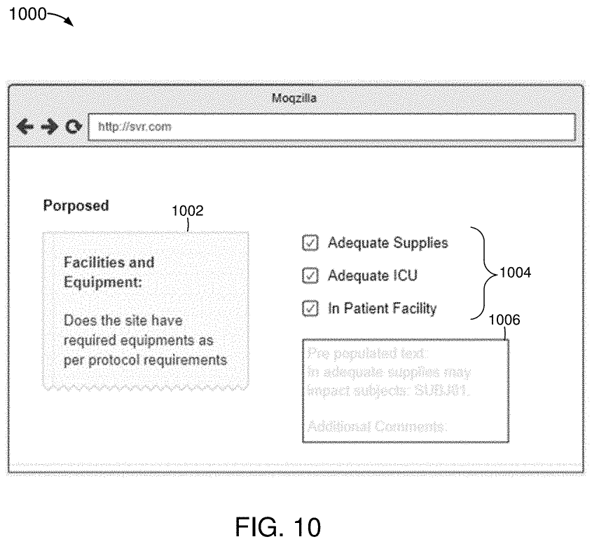

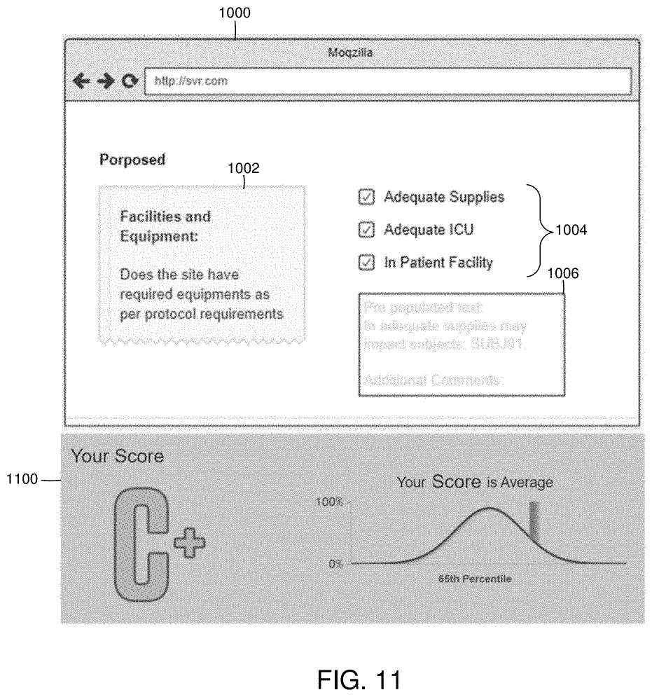

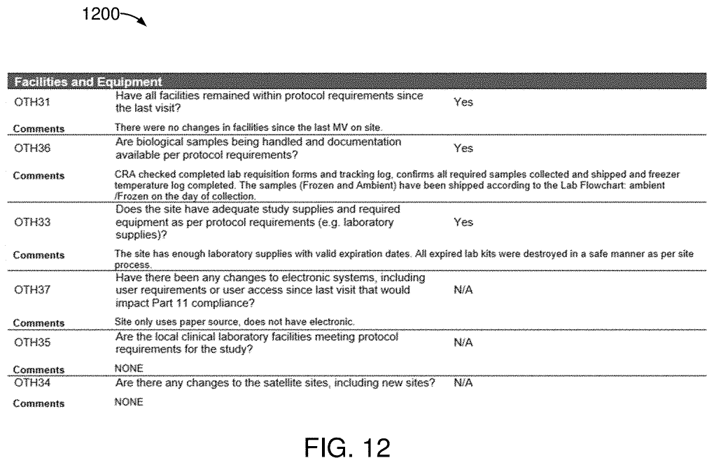

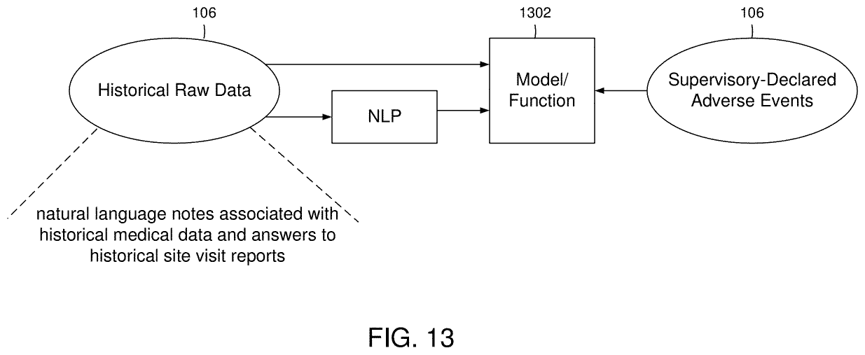

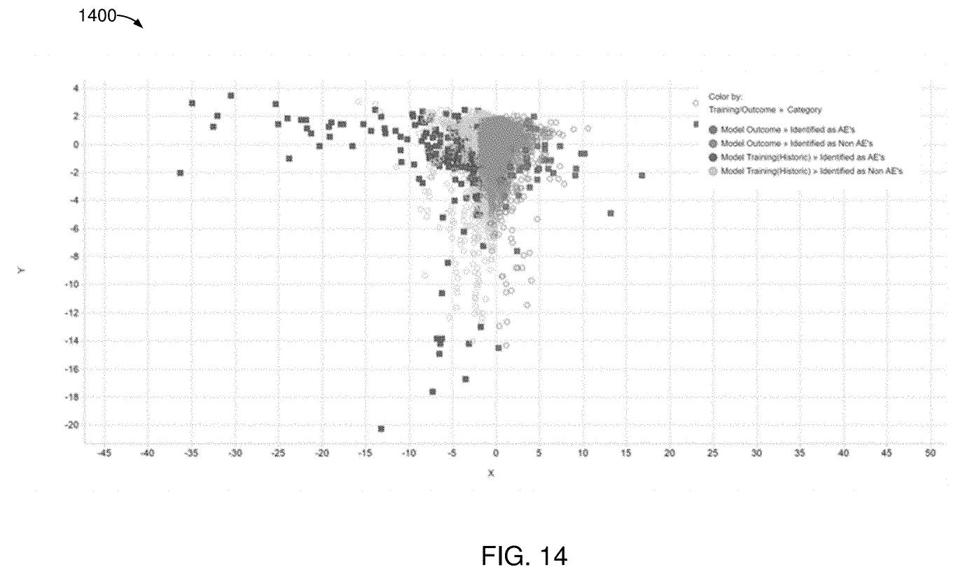

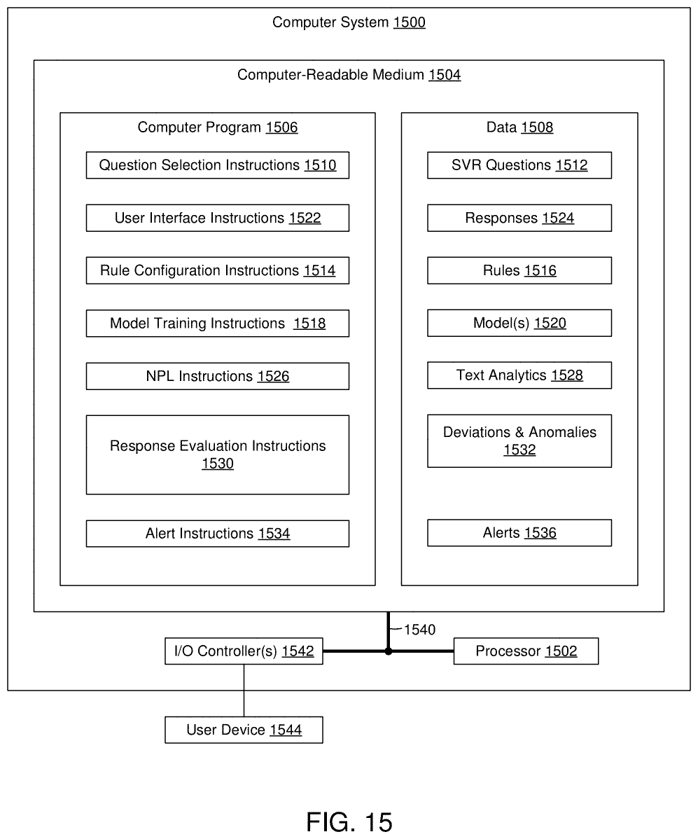

FIG. 6 is a flowchart of a method 600 of configuring and using a SVR engine.

At Error! Reference source not found.02, a configuration file (e.g., csv file, excel file, xml file, etc.) containing questions and sub-questions is generated or accessed.

At Error! Reference source not found.04, a set of questions and sub-questions is selected from the configuration file. The set of questions and sub-questions may be selected based on a site to be evaluated and/or other factors. The selected questions and sub-questions form the basis of a SVR. A SVR engine may, for example, read a pre-populated configuration file and intelligently generate questions, sub-questions, and pre-populate memo/notes section, for a given type of visit and study. The intelligence may be coded into the SVR engine based on past process experience and knowledge.

The SVR may be organized in sections. The sections may include:
Status of Clinical Study Docs;
Facilities and Equipment;
Staff Training/PI Oversight;
Adequacy of ISF;
Source Data Monitoring; and/or
IP Control and Supplies.

At 606, the selected questions and sub-questions are presented on a user device to a site monitor. The site monitor may monitor/inspect the site as per process and respond to each question and enter observations accordingly.

At Error! Reference source not found.08, responses of the site monitor are received (e.g., user-selected answers and/or natural language comments).

At 610, a measure of compliance (e.g., a compliance score) is computed for each question or a group of questions based on response(s) to the question and any sub-questions. For natural language text responses, natural language processing may be used for text classification (topic detection, sentiment analysis, etc.), and compliance of related question may be determined at least in part based on the text classification.

Compliance may be calculated for a question based on answers provided to each related sub-question and any free text comments (i.e., using NLP/AI). Different weights may be applied to each sub-question based on their relative importance. For example, a question asking for an identification of any required equipment that is missing from the site may be afforded a greater weight relative to a less important feature.

Different sections of site visit report may be co-related to determine compliance. For example, protocol deviation may be described for missing IP dosages due to hospitalization for more than 24 hours. For hospitalization spanning more than 24 hours, serious adverse events need to be reported which was missing in adverse events section. Using NLP, such compliance issues may be identified.

In an embodiment, a scoring mechanism referred to herein as Visit Report Compliance Score (VRCS) may be used. In this example, a SVR engine performs text analytics (NLP, topic modeling) and, based on responses to questions/sub-question, a compliance score is computed. The score may depend on critical and important events like protocol deviations, patient eligibility for a clinical trial, investigational Medical Product (IMP) supply and availability, and the severity score may vary based on the category of issues which in turn is determined by the unique topic modelling and sentiment analysis built into the system. For example, the scoring might be high if it finds keywords like “hospitalization,” “death”, permanent disability”, “congenital anomaly/birth defect” and flag them accordingly.

If certain contra-indicated medications were taken by subject, an alert may be generated to a project team member. Following the evaluation of each question and determining the compliance, an overall score may be computed by comparing it with all the site visit reports available for the study.

A compliance score may be useful to measure an extent to which a site is compliant with relevant rules and standards.

At 612, a determination is made as to whether a response is compliant. The determination may be based on a most recent response, alone or in combination with responses to preceding questions (e.g., a totality score). A compliance score may be compared to a threshold. The threshold may be specific to the corresponding question. If the response is non-compliant, an alert is generated at 614. The alert may be presented at the user device of the site monitor and/or other individual. The alert may include a remedial recommendation.

As an example, it may be determined whether there are any scenarios that need Serious Adverse Event (SAE) reporting, such as a patient hospitalized for more than 24 hours. If such events are not reported to ethics committee/review board within stipulated time, then raise an alert at 614.

As another example, it may be determined whether there are any events that need reporting of protocol deviations, such as IP compliance/missed dosage. If such events are not reported to ethics committee/review board within a stipulated time, then raise an alert at 614.

At 616, a performance evaluation is computed for the site, the site monitor, or an investigator associated with the study site. The performance evaluation may be based on rules applied to responses received at 608 and/or a trained model.

At 618, if the performance evaluation is poor (e.g., below a threshold and/or contrary to a rule), an alert is generated at 620. The alert may be presented at the user device of the site monitor and/or other individual. The alert may include a remedial recommendation.

At 622, the site and/or other site(s) are evaluated based on a model to determine whether the site(s) is a high-risk site (e.g., too many protocol violations and/or adverse events).

At 624, if site is determined to be high risk, an alert is generated at 626. The alert may be presented at the user device of the site monitor and/or other individual. The alert may include a remedial recommendation.

Additional examples regarding 610-626 are provided immediately below.

A missed IP dosage (protocol deviation) or missing signed informed form may result in a high non-compliance score.

Where a question related to hospitalization indicates a patient was hospitalized for more than 25 hours, and a question related to SAE has no serious adverse events reports, the issue may be flagged for further review.

An essential document that is missing from an investigator site file may adversely impact a score.

Where staff has not completed relevant training, it may adversely impact a score.

If an investigator site file (ISF) is not adequate over successive visits, it may reflect poor site monitor and/or investigator performance.

If a site monitor has not escalated an issue indicated by a response, it may indicate poor performance on the part of the site monitor.

Data points such as the number of protocol deviations, adequacy of an investigator site file (ISF) score, a clinical study documents score, or an IP controls and supplies score, may be used to identify high risk sites.

A SVR engine, as disclosed herein, may be configured to present information in a variety of windows, examples of which are provided below. Methods and systems disclosed herein are not, however, limited to the following examples.

FIG. 4 illustrates a user interface window 700 that includes a question presentation window 702, a pull-down list of user-selectable answers 704, and a comment window 706 to receive natural language comments of a user. One or more additional windows may be displayed (e.g., pop-up windows), such as in reaction to hovering a cursor over an area of window 700 and/or in response to user input at window 704 and/or window 706. FIG. 8 illustrates an example pop-up window 800. FIG. 9 illustrates an example pop-up window 900. Pop-up windows are not, however, limited to example windows 800 and 900.

FIG. 10 illustrates a user interface window 1000 that includes a question presentation window 1002, user-selectable answers 1004, and a comment window 1006 to receive natural language comments of a user.

FIG. 11 illustrates user interface window 1000 of FIG. 10 and a corresponding feedback window 1100 to present ranking or scoring information based on responses to questions presented in window 1002. Similar ranking or scoring information may be provided in response to questions presented in window 700 of FIG. 7.

FIG. 12 illustrates a portion of an example SVR 1200.

In an embodiment, a model is trained to identify adverse events from responses (i.e., user-selected answers and natural language comments) to a SVR. An example is provided below with reference to FIGS. 13 and 14.

FIG. 13 is a block diagram of a computer-trainable model/function 1302 to correlate historical raw data 1304 (e.g., text analytics extracted from historical natural language notes associated with historical medical data, and answers of historical site visit reports), with corresponding supervisor-declared adverse events 1306. Once trained, the model may be used (e.g., at 406 in FIG. 4), to detect adverse events based on responses (i.e., user-selected answers and natural language comments) to questions of a SVR.

An actual trained model is described below. Historical datasets were utilized that contain decisions around classification of data based on clinical expertise and years of process execution in the space of clinical signal detection. This unique knowledge and experience were converted to a bespoke methodology to classify scenarios as adverse events (AE) or non-adverse events (non-AE) using correlating variables such as lab values. The chosen lab tests were liver panel tests of ALT, AST, and Bilirubin. The dataset used for training contained lab values, baseline values, medical history, ongoing adverse events, and the decision taken by the subject data reviewer in the past (AE or non-AE). The resultant machine learning model was found to generate classification with near 100% accuracy and this was verified by the process subject matter experts. Verification was done with assistance of data visualization that was purpose built for this exercise to avoid any bias.

The model was tested on 6531 subject or patient visit data for the selected lab tests related to liver injury. The model classified 6378 as non-AEs and 153 as AEs. In other words, around 2-3% of them were AEs and needed expert review (subject data reviewer) and the remaining 97% do not need a medical expert to conduct manual review. The modeling procedure described herein thus provides a consistent and reliable system to identify adverse events and reduces the time and effort involved in what would otherwise be a labor-intensive process. The above-described process can be extended to other lab/vitals.

FIG. 14 illustrates a graph 1400 of AEs and non-AEs as determined by a supervisor and as determined by the model described in the preceding paragraph.

One or more features disclosed herein may be implemented in, without limitation, circuitry, a machine, a computer system, a processor and memory, a computer program encoded within a computer-readable medium, and/or combinations thereof. Circuitry may include discrete and/or integrated circuitry, application specific integrated circuitry (ASIC), a system-on-a-chip (SOC), and combinations thereof.

Information processing by software may be concretely realized by using hardware resources.

FIG. 15 is a block diagram of a computer system 1500, configured to evaluate responses to a site visit report (SVR). Computer system 1500 may be further configured to configure the SVR with questions. Computer system 1500 may be further configured to train a model for use in evaluating the responses to the SVR. Computer system 1500 may represent an example embodiment or implementation of a SVR engine described with reference to one or more other examples herein.

Computer system 1500 includes an instruction processor 1502 to execute instructions of a computer program 1506 encoded within a computer-readable medium 1504. Computer-readable medium 1504 further includes data 1508, which may be used by processor 1502 during execution of computer program 1506 and/or generated by processor 1502 during execution of computer program 1506.

Computer-readable medium 1504 may include a transitory or non-transitory computer-readable medium.

In the example of FIG. 15, computer program 1506 includes question selection instructions 1510 to cause processor 1502 to select questions and sub-questions 1512 to include in a SRV, such as described in one or more examples above.

Computer program 1506 further includes user interface instructions 1522 to cause processor 1502 to present questions 1512 on a user device and receive responses 1524 (e.g., user-selected answers and natural language comments) from the user device, such as described in one or more examples above.

Computer program 1506 further includes rule configuration instructions 1514 to cause processor 1502 to configure rules 1516 with which to process responses 1524 to questions 1512 (e.g., to detect protocol deviations), such as described in one or more examples above.

Computer program 1506 further includes model training instructions 1518 to cause processor 1502 to train a model 1520 to detect anomalies from responses 1524, such as described in one or more examples above.

Model training instructions 1518 may include instructions to cause processor 1502 to train a probabilistic topic model to detect topics from natural language notes.

Model training instructions 1518 may include instructions to cause processor 1502 to train a sentiment model to detect sentiments from natural language notes.

Computer program 1506 further includes natural language processing (NPL) instructions 1526 to cause processor 1502 to extract text analytics 1528 (e.g., sentiment analytics and/or topical analytics) from natural language comments of responses 1524, such as described in one or more examples above.

Computer program 1506 further includes response evaluation instructions 1530 to cause processor 1502 to evaluate responses 1524 based on rules 1516, analytics 1528, and model 1520 to identify deviations and anomalies 1532, such as described in one or more examples above.

Computer program 1506 further includes alert instructions 1534 to cause processor 1502 to generate alerts 1536 based on deviations and anomalies 1532, such as described in one or more examples above.

Computer system 1500 further includes communications infrastructure 1540 to communicate amongst devices and/or resources of computer system 1500.

Computer system 1500 may include one or more input/output (I/O) devices and/or controllers 1542 to interface with one or more other systems, such as to interface with a user device 1544 of a site monitor, a sponsor, and/or an investigator, such as described in one or more examples above.

Methods and system disclosed herein may be useful to determine how many sites require regulatory approvals for revised clinical study documents.

Methods and system disclosed herein may be useful to determine how many sites need training from a site monitor for revised clinical study documents.

Methods and system disclosed herein may be useful to determine how many sites have challenges in obtaining signed ICFs.

Methods and system disclosed herein may be useful to determine how many sites lack require source documentation for SAEs.

Methods and system disclosed herein may be useful to determine how many sites have challenges in dispensing and administering IP as per protocol requirements.

Methods and system disclosed herein may be useful to prioritize or tier sites based on risk and/or to identify high risk sites.

Methods and system disclosed herein may be useful to provide insight on historical performance of site monitor/site/investigator, which may be used as an input for site selection.

Methods and system disclosed herein may be useful to provide insight to assist in monitoring and mitigating risk of clinical studies.

Methods and system disclosed herein may be useful in achieving high quality and consistency in site visit report data governance with unique methods of capturing information in a SVR and generating a unique compliance score to help determine compliance risks and address such risk.

Methods and system disclosed herein may provide actionable insights to a leadership team, sponsors, or a project team regarding challenges faced at a site, which may help to mitigate risks. For example, if a source data verification (SDV) backlog is more than planned or are numerous unreported Serious adverse events (SAE), the project team may mitigate risk by extending a visit or implementing appropriate review strategies.

Methods and system disclosed herein may be useful to identify trends at a site. For example, insights like primary investigator not being available for the site monitor during monitoring visit may be flagged so that actions can be taken to address it.

Even though all activities listed in clinical operations plan have to be monitored during all the visits, they can be skipped by the site monitor to accommodate other priority review tasks based on the threshold defined for that activity. As an example, it may be permitted to omit an investigator file from review for up to three consecutive visits. If any activity has reached or is nearing a threshold limit, it may be flagged so that risks may be mitigated, such as by prioritizing it in review or making it a mandatory task in the next monitoring visit.

Methods and system disclosed herein may be useful to compute site performance by leveraging various data points captured in a SVR (e.g., across multiple visits and multiple studies conducted at a site). Data points may include insights generated by a SVR engine, feedback, inputs entered by various roles like site monitor, project team member, sponsors, and investigators. The foregoing information on site performance may lead to a unique analysis for a site, which may be useful for various stakeholders (e.g., sponsor) to provide specific and focused support. An assessment of sites may be useful in a site selection process for future clinical trials.

Methods and system disclosed herein may be useful to evaluate and ensure that access to individuals is granted and revoked based on their joining and the end date in the study conduct. For example, if there is a change in study coordinator, a SVR engine may ensure that appropriate steps are taken to revoke/grant access for the outgoing as well as the incoming site staff team members.

Methods and systems are disclosed herein with the aid of functional building blocks illustrating functions, features, and relationships thereof. At least some of the boundaries of these functional building blocks have been arbitrarily defined herein for the convenience of the description. Alternate boundaries may be defined so long as the specified functions and relationships thereof are appropriately performed. While various embodiments are disclosed herein, it should be understood that they are presented as examples. The scope of the claims should not be limited by any of the example embodiments disclosed herein.

As used herein, the word “or” indicates an inclusive list such that, for example, the list of X, Y, or Z means X or Y or Z or XY or XZ or YZ or XYZ.

As used herein, the phrase “based on” means “based at least in part on.” For example, a feature that is described as “based on condition A” may be based on both condition A and one or more other conditions.

As used herein, the words “a” or “an” indicate” at least one.

A feature or function described herein may include multiple features or functions and may be performed in conjunction with other features or functions.

The description and drawings represent example configurations and do not represent all the implementations within the scope of the claims. For example, operations may be rearranged, combined, or otherwise modified. Also, structures and devices may be represented in the form of block diagrams to represent the relationship between components and avoid obscuring the described concepts. Similar components or features may have the same name but may have different reference numbers corresponding to different figures.

Some modifications to the disclosure may be readily apparent to those skilled in the art, and the principles defined herein may be applied to other variations without departing from the scope of the disclosure. Thus, the disclosure is not limited to the examples and designs described herein but is to be accorded the broadest scope consistent with the principles and novel features disclosed herein.

What is claimed is:

1. A machine-implemented method, comprising:
presenting of a site visit report in a sequential fashion on a user device during a site visit;
receiving responses to the questions via the user device, wherein the responses include user-selectable answers and natural language notes of a user;
evaluating each response as it is received from the user device to detect an anomaly in the clinical trial site visit, including evaluating the user-selected answers and the text analytics based on a combination of pre-configured rules and a computer-trained model, wherein the anomaly includes a protocol deviation and/or an adverse event, and wherein the text analytics includes sentiment analytics and/or topical analytics;
determining if the detected anomaly is already identified as an anomaly in the site visit report; and
generating an alert, during the site visit, if the detected anomaly is not already identified as an anomaly in the site visit report, wherein the alert includes a recommendation to resolve the anomaly.

2. The method of claim 1, further comprising:
selecting the questions to include in the site visit report based on features of a site and an associated clinical study;
configuring the rules to identify anomalies in the responses; and
training the model to correlate historical medical data with supervisor-identified anomalies in the historical medical data, wherein the historical medical data includes patient data, trial data, and laboratory test results.

3. The method of claim 1, wherein:
the evaluating comprises computing a compliance score for each response and detecting the anomaly when the compliance score exceeds a threshold.

4. The method of claim 1, wherein:
the generating an alert comprises ranking the detected anomaly based on a safety-related risk factor associated with the anomaly, during the site visit.

5. The method of claim 1, further comprising:
training a probabilistic topic model to detect topics from historical natural language notes associated with historical medical data; and
training a sentiment model to detect sentiments from the historical natural language notes;
wherein the evaluating comprises computing the text analytics with the probabilistic topic model and the sentiment model.

6. The method of claim 1, further comprising:
training the model to correlate text analytics extracted from historical natural language notes associated with historical medical data, and answers of historical site visit reports, with corresponding supervisor-declared adverse events;
wherein the evaluating comprises evaluating the text analytics and at least a subset of the responses with the trained model.

7. The method of claim 1, further comprising:
evaluating multiple site visit reports in combination with one another to detect a pattern of anomalies.

8. A non-transitory computer readable medium encoded with a computer program that comprises instructions to cause a processor to:
present questions of a site visit report in a sequential fashion on a user device during a site visit;
receive responses to the questions via the user device, wherein the responses include user-selectable answers and natural language notes of a user;
evaluate each response as it is received from the user device to detect an anomaly in the site visit, including to evaluate the user-selected answers and text analytics of the natural language notes based on a combination of pre-configured rules and a computer-trained model, wherein the anomaly includes a protocol deviation and/or an adverse event, and wherein the text analytics includes sentiment analytics and/or topical analytics;
determine if the detected anomaly is already identified as an anomaly in the site visit report; and
generate an alert, during the site visit, if the detected anomaly is not already identified as an anomaly in the site visit report, wherein the alert includes a recommendation to resolve the anomaly.

9. The non-transitory computer readable medium of claim 8, further including instructions to cause the processor to:
select the questions to include in the site visit report based on features of a site and an associated clinical study;
configure the rules to identify anomalies in the responses; and
train the model to correlate historical medical data with supervisor-identified anomalies in the historical medical data, wherein the historical medical data includes patient data, trial data, and laboratory test results.

10. The non-transitory computer readable medium of claim 8, further including instructions to cause the processor to:
compute a compliance score for each of the responses; and
detect the anomaly when the compliance score exceeds a threshold.

11. The non-transitory computer readable medium of claim 8, further including instructions to cause the processor to:
rank the detected anomaly based on a safety-related risk factor associated with the anomaly, during the site visit.

12. The non-transitory computer readable medium of claim 8, further including instructions to cause the processor to:
train a probabilistic topic model to detect topics from historical natural language notes associated with historical medical data;
train a sentiment model to detect sentiments from the historical natural language notes; and
compute the text analytics with the probabilistic topic model and the sentiment model.

13. The non-transitory computer readable medium of claim 8, further including instructions to cause the processor to:
train the model to correlate text analytics extracted from historical natural language notes associated with historical medical data, and answers of historical site visit reports, with corresponding supervisor-declared adverse events; and
evaluate the text analytics and at lease a subset of the responses with the trained model.

14. The non-transitory computer readable medium of claim 8, further including instructions to cause the processor to:
evaluate multiple site visit reports in combination with one another to detect a pattern of deviations and/or anomalies.

15. An apparatus, comprising a processor and memory configured to:
present questions of a site visit report in a sequential fashion on a user device during a site visit;
receive responses to the questions via the user device, wherein the responses include user-selectable answers and natural language notes of a user;
evaluate each response as it is received from the user device to detect an anomaly in the site visit, including to evaluate the user-selected answers and text analytics of the natural language notes based on a combination of pre-configured rules and a computer-trained model, wherein the anomaly includes a protocol deviation and/or an adverse event, and wherein the text analytics includes sentiment analytics and/or topical analytics;
determine if the detected anomaly is already identified as an anomaly in the site visit report; and
generate an alert, during the site visit, if the detected anomaly is not already identified as an anomaly in the site visit report, wherein the alert includes a recommendation to resolve the anomaly.

16. The non-transitory computer readable medium of claim 8, further including instructions to cause the processor to:
select the questions to include in the site visit report based on features of a site and an associated clinical study;
configure the rules to identify anomalies in the responses; and
train the model to correlate historical medical data with supervisor-identified anomalies in the historical medical data, wherein the historical medical data includes patient data, trial data, and laboratory test results.

17. The non-transitory computer readable medium of claim 8, further including instructions to cause the processor to:
compute a compliance score for each of the responses; and
detect the anomaly when the compliance score exceeds a threshold.

18. The non-transitory computer readable medium of claim 8, further including instructions to cause the processor to:
rank the detected anomaly based on a safety-related risk factor associated with the anomaly, during the site visit.

19. The non-transitory computer readable medium of claim 8, further including instructions to cause the processor to:
train a probabilistic topic model to detect topics from historical natural language notes associated with historical medical data;
train a sentiment model to detect sentiments from the historical natural language notes; and
compute the text analytics with the probabilistic topic model and the sentiment model.

20. The non-transitory computer readable medium of claim 8, further including instructions to cause the processor to:
train the model to correlate text analytics extracted from historical natural language notes associated with historical medical data, and answers of historical site visit reports, with corresponding supervisor-declared adverse events; and
evaluate the text analytics and at lease a subset of the responses with the trained model.

21. The non-transitory computer readable medium of claim 8, further including instructions to cause the processor to:
evaluate multiple site visit reports in combination with one another to detect a pattern of deviations and/or anomalies.
